(19) United States
(12) Patent Application Publication
(10) Pub. No.: US 2023/0260613 A1
(43) Pub. Date: Aug. 17, 2023

(54) AI-DRIVEN CARE PLANNING USING SINGLE-SUBJECT MULTI-MODAL INFORMATION

(71) Applicant: Siemens Healthcare GmbH, Erlangen (DE)

(72) Inventor: Venkata Veerendranadh Chebrolu, Rochester, MN (US)

(21) Appl. No.: 18/152,242

(22) Filed: Jan. 10, 2023

Related U.S. Application Data
(60) Provisional application No. 63/309,053, filed on Feb. 11, 2022.

(51) Int. Cl. G16H 20/00; G16H 10/60; G06T 7/00

(52) U.S. Cl. CPC G16H 20/00; G16H 10/60; G06T 7/0012; G06T 2207/20081

(57) ABSTRACT

A system and method include determination of multi-modal data associated with a subject, the multi-modal data including image data of the subject, input of the multi-modal data to a first trained clustering model to determine a cluster for the subject, determination of a proposed treatment for the subject, and input of the multi-modal data, the cluster and the treatment to a second trained model, where the second trained model outputs a probability associated with a treatment outcome in response to the input multi-modal data, cluster and treatment.

### CROSS-REFERENCE TO RELATED APPLICATIONS

This application claims priority to U.S. Provisional Patent Application No. 63/309,053, filed Feb. 11, 2022, the disclosure of which is incorporated herein by reference for all purposes.

### BACKGROUND

Conventional medical imaging systems are capable of generating high-quality images of internal structures and processes. Medical imaging is therefore commonly used for disease prevention, diagnosis, and treatment planning. Many types of medical imaging exist, including x-ray imaging, computed tomography (CT) imaging, positron emission tomography (PET) imaging, single photon emission computed tomography (SPECT), ultrasound, and magnetic resonance (MR) imaging.

Additional data is often used in conjunction with medical images to perform prevention, diagnosis, and treatment planning. This data may include historical and/or current test results (i.e., blood, urine, tissue), lifestyle information, demographic information, family history, vital sign monitoring, etc. It is often difficult to reach reliable conclusions based on this complex and interdependent data, particularly in the case of treatment planning.

For example, current guidelines for many types of care are based on the population/group to which a patient is deemed to belong, rather than on a purely individualized assessment of the patient. These guidelines are developed based on cross-sectional and longitudinal studies of such groups, which may show differences (expressed as a mean₊ standard deviation of some parameter) in outcomes resulting from various treatments, i.e., monitoring regimes and/or clinical pathways. Accordingly, a group to which a patient belongs is determined, and a clinical pathway for treatment is determined by identifying the pathways which provided the most desirable outcomes for that group.

There is significant interest and need for more-individualized inferences, decisions, advice, and treatment planning. Clinical research into blood, tissue, and urine-based genomic and proteomic methods for achieving such individualization is underway. However, such methods do not incorporate multi-modal patient-specific information which also includes medical images.

### BRIEF DESCRIPTION OF THE DRAWINGS

FIG. 1 illustrates a subject and associated multi-modal clinical data according to some embodiments.

FIG. 2 is a block diagram of an architecture including two trained models for determining probabilities of clinical outcomes based on multi-modal data and a proposed clinical pathway according to some embodiments.

FIG. 3 is a flow diagram of a process to train a clustering model and a model to determine probabilities of clinical outcomes based on multi-modal data and a proposed clinical pathway according to some embodiments.

FIG. 4 is a block diagram illustrating training of a clustering model based on multi-modal data according to some embodiments.

FIG. 5 is a block diagram illustrating determination of a cluster for each of a plurality of test subjects using a trained clustering model according to some embodiments.

FIG. 6 is a block diagram illustrating training of a model to determine probabilities of clinical outcomes based on multi-modal data and a proposed clinical pathway according to some embodiments.

FIG. 7 is a block diagram illustrating usage of a cloud service by client devices to determine probabilities of clinical outcomes based on multi-modal data and a proposed clinical pathway according to some embodiments.

FIG. 8 is a flow diagram of a process to use trained models to determine probabilities of clinical outcomes based on multi-modal data and a proposed clinical pathway according to some embodiments.

FIG. 9 is a block diagram of a system to train models according to some embodiments.

FIG. 10 is a block diagram of a magnetic resonance imaging system according to some embodiments.

### DETAILED DESCRIPTION

The following description is provided to enable any person in the art to make and use the described embodiments and sets forth the best mode contemplated for carrying out the described embodiments. Various modifications, however, will remain apparent to those in the art.

Some embodiments apply trained algorithms to patient-specific multi-modal data to provide individualized assessments of outcomes for various treatments. The multi-modal data may consist of, but is not limited to, image data (e.g., MR, ultrasound), demographic data, lab tests (e.g., protein-specific antigen (PSA) measurements), genomic data, patient symptoms, and case reports. The multi-modal data may include data generated based on other multi-modal data, such as region labels determined by segmentation of image data as is known in the art.

According to some embodiments, a clustering model is trained to assign subjects to respective clusters based on multi-modal data of many test subjects. Another model is trained to output a probability for each of one or more clinical outcomes based on, for each test subject, multi-modal data, a cluster determined by the trained clustering model, a clinical pathway pursued by the test subject, and flags representing the presence or absence of the one or more clinical outcomes.

The trained models may be used to evaluate a proposed clinical pathway for a patient. For example, multi-modal data associated with the patient is input to the trained clustering model to determine a cluster associated with the patient. The multi-modal data, the determined cluster, and a proposed clinical pathway are then input to the other trained model to determine a probability that the patient will experience each of the one or more clinical outcomes if the patient pursues the proposed clinical pathway.

The trained models may be used as described above to determine new probabilities when new multi-modal data is generated for the patient. Using new multi-modal data may result in determination of a new cluster for the patient, which may affect the newly-determined probabilities. If such newly-determined probabilities are unfavorable, a clinician may propose a new clinical pathway and determine the one or more outcome probabilities based thereon.

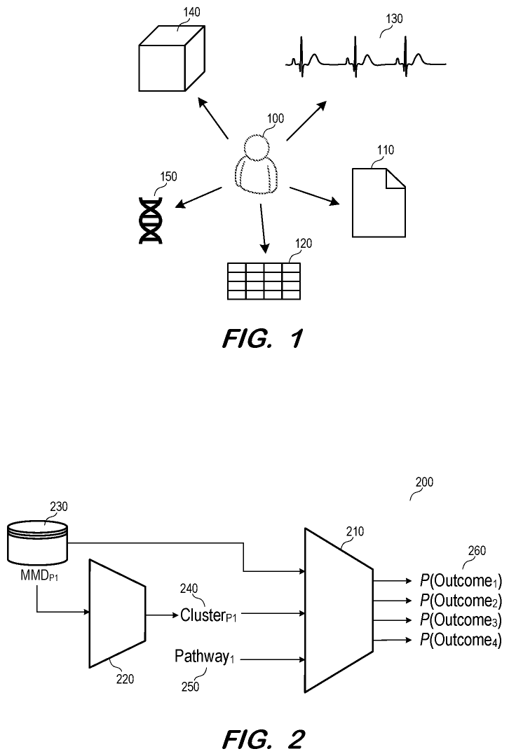

FIG. 1 illustrates the disparate types of data which may be associated with patient 100 and used for treatment planning according to some embodiments. For example, patient 100 may be associated with medical reports 110 which provide physician notes and conclusions as well as physiological data, lab results 120 which provide values of physiological parameters such as levels of certain chemical markers, time series data such as EKGs or respiratory cycle waveforms, one or more three-dimensional images 140, and genomic data 150. The data of FIG. 1 are merely representative and not intended to limit the type, format or volume of multi-modal data which may be used in some embodiments. As is known in the art, multi-modal data associated with patient 100 may change over time as patient 100 is subjected to subsequent testing, imaging, and monitoring.

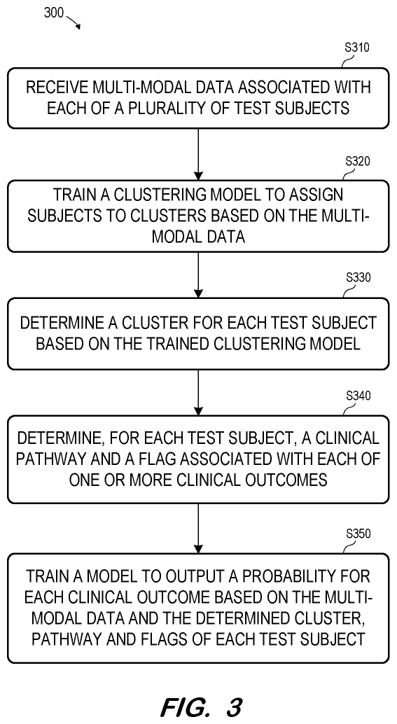

FIG. 2 illustrates architecture 200 according to some embodiments. Architecture 200 may be operated to determine probabilities of various clinical outcomes for a given patient based on multi-modal data associated with the patient and a proposed clinical pathway. Architecture 200 includes trained model 210 and trained model 220, the training of which according to some embodiments will be described below.

For example, multi-modal data 230 associated with patient P1 is input to trained clustering model 220. Multi-modal data 230 may consist of any suitable types of data described herein or otherwise known. Clustering model 220 operates per its training to infer cluster identifier 240.

Multi-modal data 230 and cluster identifier 240 are input to trained model 210. Also input to trained model 210 is pathway identifier 250, which identifies a clinical pathway used to treat (or not treat) a particular condition. A clinical pathway may comprise a particular drug regimen, chemo or radiation therapy regimen, diet, and exercise regimen and/or any combination thereof. Trained model 210 operates on these inputs to generate probabilities 260. Each of probabilities 260 is a probability of the occurrence of a given outcome if patient P1 undertakes identified clinical pathway 250. Examples of outcomes for which probabilities may be determined include but are not limited to remission, survival for more than 5 years, maintenance of life quality, ability to perform specific tasks, etc.

Models 210 and 220 may comprise any type of learning model, network or algorithm that is or becomes known. Broadly, a model may comprise an arrangement of logical nodes which receive input, change internal state according to that input, and produce output depending on the input and internal state. The output of certain nodes is connected to the input of other nodes to form a directed and weighted graph. The weights as well as the functions that compute the internal state can be modified via training as will be described below. Models 210 and 220 may comprise one or more types of artificial neural network that are or become known, including but not limited to convolutional neural networks, recurrent neural networks, long short-term memory networks, deep reservoir computing and deep echo state networks, deep belief networks, and deep stacking networks.

FIG. 3 comprises a flow diagram of process 300 to train models according to some embodiments. Process 300 and the other processes described herein may be performed using any suitable combination of hardware, software, or other means. Software embodying these processes may be stored by any non-transitory tangible medium, including but not limited to a fixed disk, a DVD, a Flash drive, a cloud, or a magnetic tape.

Initially, at S310, multi-modal data associated with each of a plurality of test subjects is received. The data may be received from disparate public and/or private data repositories and may be anonymized to protect individual privacy. The multi-modal data for each test subject may include image data from one or more imaging modalities, lab results, clinician reports, test results, demographic data, lifestyle data, genomic data, and any other health-related data of any format that is or becomes known.

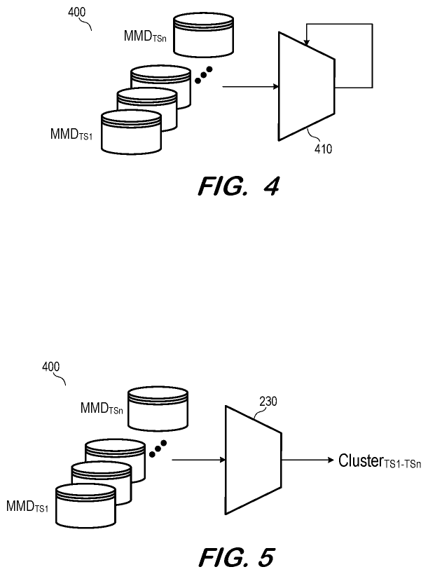

Next, at S320, the multi-modal data is used to train a clustering model to assign a subject to a cluster. FIG. 4 illustrates S320 according to some embodiments. As shown, multi-modal data 400 is associated with each of test subjects TS1 through TSn. Multi-modal data 400 is input to clustering model 410, which may execute an unsupervised learning algorithm to define clusters based thereon. As is known in the art, clustering model 410 may generate features based on the multi-modal data 400 of each of test subjects TS1 through TSn such that each test subject is associated with a multi-dimensional vector of features. During training, parameters of clustering model 410 are modified to generate mappings between features vectors and clusters based on similarities and differences between the feature vectors.

A cluster for each test subject is determined based on the trained clustering model at S330. In one embodiment illustrated in FIG. 5, multi-modal data 400 is input to the trained clustering model (now labeled as model 230) to generate, based on the trained parameters thereof, a cluster identifier associated with each of the test subjects. For example, multi-modal data_{TS1} is input to trained clustering model 230 to generate cluster identifier Cluster _{TS1}.

At S340, a potential clinical pathway is determined for each subject based on inputs from clinicians and subject preferences. The clinical pathway determined for a test subject may be a clinical pathway actually undertaken by the test subject. Moreover, for each test subject, a flag is determined for each of one or more clinical outcomes. The flag for a clinical outcome indicates whether or not the test subject experienced the clinical outcome. For example, if a test subject survived more than 5 years from commencement of the pathway, an associated flag may be set to value ‘1’. However, if the test subject did not enter remission, a flag associated with remission is set to value ‘0’.

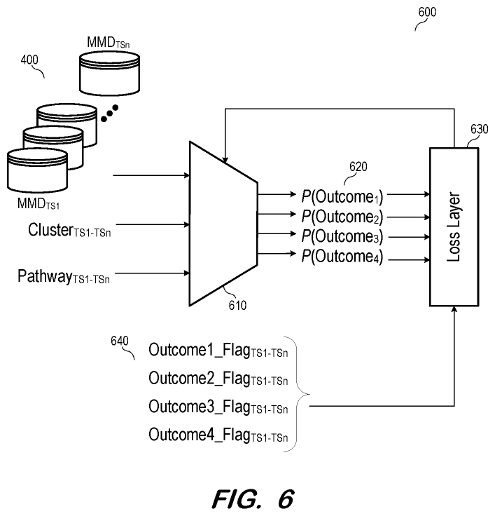

A model is then trained at S350 to output a probability for each clinical outcome. The model is trained based on the multi-modal data, cluster, pathway, and flag(s) determined for each test subject. FIG. 6 illustrates training architecture 600 for use at S350 according to some embodiments. Generally, architecture 600 trains model 610 to implement a function. The training is performed by inputting multi-modal data 400 associated with each test subject, cluster identifiers determined for each test subject and pathways determined for each test subject to model 610 and comparing output 620 against corresponding ground truths 640 associated with each test subject. The ground truths 640 are flags indicating, for each test subject and corresponding pathway, the presence of absence of four different clinical outcomes.

More specifically, according to some embodiments, data associated with a batch of test subjects is input to model 610. For example, multi-modal data 400 associated with test subjects TS1 through TS100 (i.e., MMD_{TS1}-MMD_{TS100}), cluster identifiers Cluster_{TS1}-Cluster_{TS100} pathways Pathway_{TS1}-Pathway_{TS100} may be input to model 610. Model 610 operates according to its initial configuration to output a corresponding set of inferred outcome probabilities 620 for each test subject of the batch. Loss layer 630 determines a loss by comparing the batch of outcome probabilities 620 with corresponding ones of ground truths 640. The determined loss reflects a difference between the batch of outcome probabilities 620 and corresponding ones of ground truths 640.

As is known in the art, the loss is back-propagated to model 610 in order to modify model 610 in an attempt to minimize the loss. The process repeats with respect to a different batch of test subjects and model 610 is iteratively modified in this manner until the loss reaches acceptable levels or training otherwise terminates (e.g., due to time constraints or to the loss asymptotically approaching a lower bound). At this point, model 610 is considered trained. Trained model 610 may be subjected to testing at S350. If the performance of trained model 610 is not sufficient, model 610 may be re-trained using different training parameters. The foregoing is then repeated for other clinical pathways.

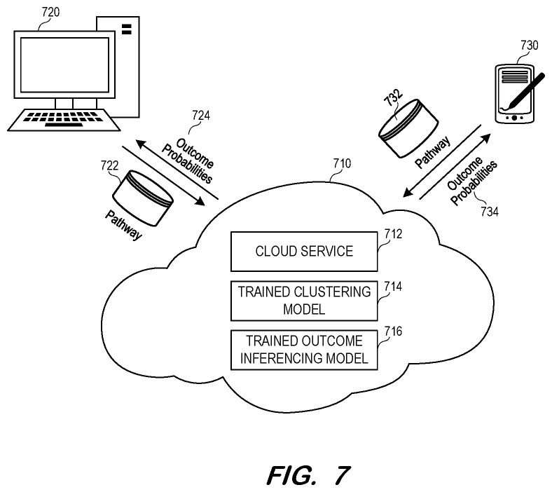

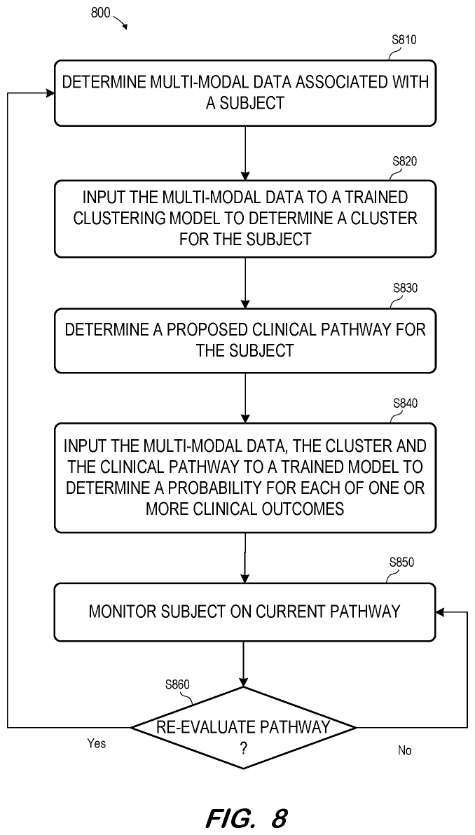

FIG. 7 is a block diagram illustrating usage of a cloud-based service by client devices to determine probabilities of clinical outcomes based on multi-modal data and a proposed clinical pathway according to some embodiments.

Cloud platform 710 may comprise any architecture for providing services (e.g., Software-as-a-Service) using cloud-based resources, and/or other systems which apportion computing resources elastically according to demand, need, price, and/or any other metric. Due to storage and processing power available via a cloud-based architecture, it may be beneficial to train machine learning models using such cloud-based resources.

Cloud service 712 may comprise a service which allows subscribing clients to define machine learning models, training data, and training algorithms for training machine learning models. Cloud service 712 may also respond to requests for inference by receiving data, inputting the received data to trained models, and returning inferred model outputs. According to the present example, clustering model 714 an outcome inferencing model 716 have been trained as described above.

Client systems 720 and 730 may comprise any computing systems capable of transmitting data to and receiving data from cloud service 712. Each of client systems 730 and 730 may be in communication with a local database system (not shown) storing patient data, a local imaging system for generating patient images, or other source of patient data. According to some embodiments, client systems 720 and 730 execute Web applications (e.g., within virtual machines of respective Web browsers) to request inferences and receive responses from cloud service 712.

For example, client system 720 may transmit patient multi-modal data 722 and a proposed clinical pathway to cloud service 712. As described above, cloud service 712 may input multi-modal data 722 to trained clustering model 714 to determine an associated cluster. Next, the cluster, the pathway, and multi-modal data 722 are input to trained outcome inferencing model 714 to generate a probability for each of one or more outcomes. Outcome probabilities 724 are then returned to computing system 720.

Client system 730 may also transmit patient multi-modal data 732 and a proposed clinical pathway to cloud service 712. Data 732 and/or the proposed pathway may differ from that transmitted by system 720 and may be associated with a same or different patient. Cloud service 712 inputs multi-modal data 732 to trained clustering model 714 to determine an associated cluster, and the cluster, the pathway received from system 730, and multi-modal data 732 are input to trained outcome inferencing model 714 to generate a probability for each of the one or more outcomes. These outcome probabilities 734 are then returned to computing system 730.

FIG. 8 is a flow diagram of process 800 to use a trained clustering model and another trained model to determine probabilities of various clinical outcomes for a given patient based on multi-modal data associated with the patient and a proposed clinical pathway. Initially, multi-model data associated with a subject, i.e., the patient, is determined at S810. As described herein, the multi-modal data may be acquired at S810 from disparate sources, and some of the multi-modal data may have been generated at different times than others of the multi-modal data.

The multi-modal data is input to a trained clustering model (e.g., trained clustering model 220) to determine a cluster associated with the subject. Next, at S830, a proposed clinical pathway for the subject is determined. The proposed clinical pathway may be selected from among the different clinical pathways used in the training of a model such as model 610.

The multi-modal data, the determined cluster, and the proposed clinical pathway are input to a trained model (e.g., trained model 210). The trained model operates on these inputs to determine a probability for each of one or more clinical outcomes. The identity and number of clinical outcomes depends on the ground truths used to train the model.

Flow proceeds to S850 to monitor the subject on the current clinical pathway. Such monitoring may include the collection over time of new multi-modal data associated with the subject. At S860, it is determined whether or not to re-evaluate the clinical pathway. If the determination is negative, flow returns to S850 to continue monitoring the subject. Alternatively, it may be determined that newly-acquired multi-modal data associated with the subject represents a significant physiological change from the time at which S840 was last executed. Accordingly, it may be determined at S860 to re-evaluate the pathway and flow therefore proceeds from S860 to S810 to determine multi-modal data associated with the subject, which includes the new multi-modal data collected at S850.

It should be noted that, due to the new multi-modal data, the cluster determined at S820 may differ from a previously-determined cluster for the subject. Next, the same or a new clinical pathway may be proposed at S830, and the new input data, the potentially-new cluster and the potentially-new pathway are input to the trained model at S840. Flow may then proceed as described above based on the resulting outcome probabilities. Accordingly, process 800 may be used to select a clinical pathway and to efficiently re-evaluate the selected or other clinical pathways in response to changes to a patient condition.

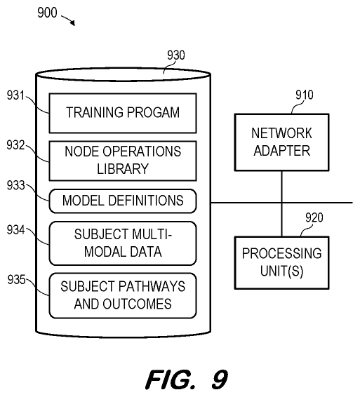

FIG. 9 illustrates computing system 900 according to some embodiments. System 900 may comprise a computing system to facilitate the design and training of machine learning models. Computing system 900 may comprise a standalone system, or one or more elements of computing system 900 may be provided by cloud-based resources.

System 900 includes network adapter 910 to communicate with external devices via a network connection. Processing unit(s) 920 may comprise one or more processors, processor cores, or other processing units to execute processor-executable program code. In this regard, storage system 930, which may comprise one or more memory devices (e.g., a hard disk drive, a solid-state drive), stores processor-executable program code of training program 931 which may be executed by processing unit(s) 920 to train a model as described herein.

Training program 931 may utilize node operations library 932, which includes program code to execute various operations associated with node operations as defined in node operations library 932. According to some embodiments, computing system 900 provides interfaces and development software (not shown) to enable development of training program 931 and generation of model definitions 933. Storage device 930 also includes training data consisting of training subject multi-modal data 934 and pathways and associated outcomes 935 for each training subject.

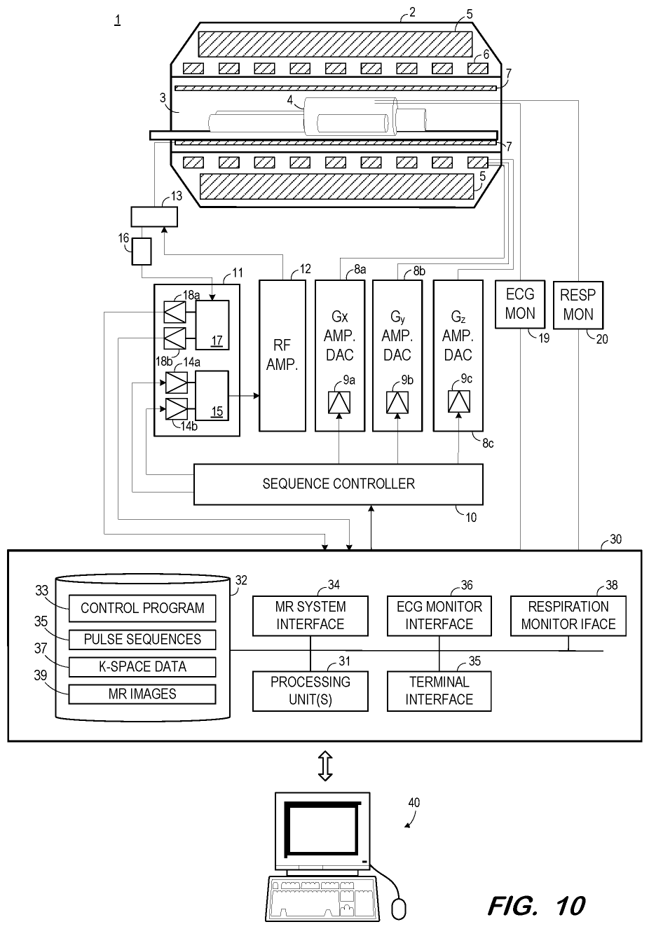

FIG. 10 illustrates MR system 1 according to some embodiments. MR system 1 may be used to generate images for inclusion with subject-specific multi-modal data as described herein. MR system 1 includes MR chassis 2, which defines bore 3 in which patient 4 is disposed. MR chassis 2 includes polarizing main magnet 5, gradient coils 6 and RF coil 7 arranged about bore 3. According to some embodiments, polarizing main magnet 5 generates a uniform main magnetic field (B0) and RF coil 7 emits an excitation field (B1).

According to MR techniques, a substance (e.g., human tissue) is subjected to a main polarizing magnetic field (i.e., B0), causing the individual magnetic moments of the nuclear spins in the substance to process about the polarizing field in random order at their characteristic Larmor frequency, in an attempt to align with the field. A net magnetic moment Mz is produced in the direction of the polarizing field, and the randomly-oriented magnetic components in the perpendicular plane (the x-y plane) cancel out one another.

The substance is then subjected to an excitation field (i.e., B1) created by emission of a radiofrequency (RF) pulse, which is in the x-y plane and near the Larmor frequency, causing the net aligned magnetic moment Mz to rotate into the x-y plane so as to produce a net transverse magnetic moment Mt, which is rotating, or spinning, in the x-y plane at the Larmor frequency. The excitation field is terminated, and signals are emitted by the excited spins as they return to their pre-excitation field state. The emitted signals are detected, digitized and processed to reconstruct an image or a spectrum using one of many well-known MR techniques.

Gradient coils 6 produce magnetic field gradients Gx, Gy, and Gz which are used for position-encoding NMR signals. The magnetic field gradients Gx, Gy, and Gz distort the main magnetic field in a predictable way so that the Larmor frequency of nuclei within the main magnetic field varies as a function of position. Accordingly, an excitation field B1 which is near a particular Larmor frequency will tip the net aligned moment Mz of those nuclei located at field positions which correspond to the particular Larmor frequency, and signals will be emitted only by those nuclei after the excitation field B1 is terminated.

Gradient coils 6 may consist of three windings, for example, each of which is supplied with current by an amplifier 8a-8c in order to generate a linear gradient field in its respective Cartesian direction (i.e., x, y, or z). Each amplifier 8a-8c includes a digital-analog converter 9a-9c which is controlled by a sequence controller 10 to generate desired gradient pulses at prescribed times.

Sequence controller 10 also controls the generation of RF pulses by RF system 11 and RF power amplifier 12. RF system 11 and RF power amplifier 12 are responsive to a scan prescription and direction from sequence controller 10 to produce RF pulses of the desired frequency, phase, and pulse amplitude waveform. The generated RF pulses may be applied to the whole of RF coil 7 or to one or more local coils or coil arrays. RF coil 7 converts the RF pulses emitted by RF power amplifier 12, via multiplexer 13, into a magnetic alternating field to excite the nuclei and align the nuclear spins of the object to be examined or the region of the object to be examined. As mentioned above, RF pulses may be emitted in a magnetization preparation step to enhance or suppress certain signals.

The RF pulses are represented digitally as complex numbers. Sequence controller 10 supplies these numbers in real and imaginary parts to digital-analog converters 14a-14b in RF system 11 to create corresponding analog pulse sequences. Transmission channel 15 modulates the pulse sequences with a radio-frequency carrier signal having a base frequency corresponding to the resonance frequency of the nuclear spins in the volume to be imaged.

RF coil 7 both emits radio-frequency pulses as described above and scans the alternating field which is produced because of precessing nuclear spins, i.e., the nuclear spin echo signals. The received signals are received by multiplexer 13, amplified by RF amplifier 16 and demodulated in receiving channel 17 of RF system 11 in a phase-sensitive manner. Analog-digital converters 18a and 18b convert the demodulated signals into digitized real and imaginary components.

Electrocardiograph (“ECG”) monitor 19 acquires ECG signals from electrodes placed on patient 4 and respiratory monitor 20 acquires respiratory signals from a respiratory bellows or other respiratory monitoring device. Such physiological signals may be used by sequence controller 10 to synchronize, or “gate”, transmitted RF pulses of a pulse sequence based on the heartbeat and/or respiration of patient 4.

Computing system 30 receives the digitized real and imaginary components from analog-digital converters 18a and 18b and may process the components according to known techniques. Such processing may, for example, include reconstructing two-dimensional or three-dimensional images by performing a Fourier transformation of raw k-space data, performing other image reconstruction techniques such as iterative or back-projection reconstruction techniques, applying filters to raw k-space data or to reconstructed images, generating functional magnetic resonance images, calculating motion or flow images, and generating a chemical shift vs. magnitude spectrum.

System 30 may comprise any general-purpose or dedicated computing system. Accordingly, system 30 includes one or more processing units 31 (e.g., processors, processor cores, execution threads, etc.) configured to execute processor-executable program code to cause system 30 to operate as described herein, and storage device 32 for storing the program code. Storage device 32 may comprise one or more fixed disks, solid-state random access memory, and/or removable media (e.g., a thumb drive) mounted in a corresponding interface (e.g., a USB port).

One or more processing units 31 may execute program code of control program 33 to provide instructions to sequence controller 10 via MR system interface 34. For example, sequence controller 10 may be instructed to initiate a desired pulse sequence of pulse sequences 35. In particular, sequence controller 10 may be instructed to control the switching of magnetic field gradients via amplifiers 8a-8c at appropriate times, the transmission of radio-frequency pulses having a specified phase and amplitude at specified times via RF system 11 and RF amplifier 12, and the readout of the resulting MR signals. The timing of the various pulses of a pulse sequence may be based on physiological data received by ECG monitor interface 36 and/or respiratory monitor 38.

Storage device 32 stores raw k-space data 37 and MR images 39 generated therefrom. Such data and images may be provided to terminal 40 via terminal interface 35 of system 30. Terminal interface 35 may also receive input from terminal 40, which may be used to provide commands to control program 33 to initiate imaging. Terminal 40 may transmit MR images 39, other data and a proposed clinical pathway to a service such as cloud service 712 to receive outcome probabilities as described herein. Terminal 40 may comprise a display device and an input device coupled to system 30. In some embodiments, terminal 40 is a separate computing device such as, but not limited to, a desktop computer, a laptop computer, a tablet computer, and a smartphone.

Each element of system 1 may include other elements which are necessary for the operation thereof, as well as additional elements for providing functions other than those described herein. Storage device 32 may also store data and other program code for providing additional functionality and/or which are necessary for operation of system 30, such as device drivers, operating system files, etc.

Those in the art will appreciate that various adaptations and modifications of the above-described embodiments can be configured without departing from the claims. Therefore, it is to be understood that the claims may be practiced other than as specifically described herein.

What is claimed is:

1. A system comprising:
a memory storing processor-executable program code; and
a processing unit to execute the program code to cause the system to:
determine multi-modal data associated with a subject, the multi-modal data including image data of the subject;
input the multi-modal data to a first trained clustering model to determine a cluster for the subject;
determine a proposed treatment for the subject; and
input the multi-modal data, the cluster and the treatment to a second trained model, where the second trained model outputs a probability associated with a treatment outcome in response to the input multi-modal data, cluster and treatment.

2. A system according to claim 1, wherein the probability is a probability that the treatment will result in the treatment outcome for the subject.

3. A system according to claim 2, wherein the second trained model outputs a second probability associated with a second treatment outcome in response to the input multi-modal data, cluster and treatment, and
wherein the second probability is a probability that the treatment will result in the second treatment outcome for the subject.

4. A system according to claim 1, wherein the first trained clustering model is trained by:
receiving training multi-modal data associated with each of a plurality of test subjects; an
training the first clustering model using the training multi-modal data, and
wherein the second trained model is trained by:
determining a cluster for each test subject based on the trained first model;
determining, for each test subject, a treatment and a flag associated with the treatment outcome; and
training the second model to output a probability for the treatment outcome based on the multi-modal data and the determined cluster, treatment and flag of each test subject.

5. A system according to claim 4, wherein the second trained model outputs a second probability associated with a second treatment outcome in response to the input multi-modal data, cluster and treatment,
wherein the probability is a probability that the treatment will result in the treatment outcome for the subject,
wherein the second probability is a probability that the treatment will result in the second treatment outcome for the subject, and
wherein the second trained model is trained by:
determining, for each test subject, a second flag associated with the second treatment outcome; and
training the second model to output probabilities for the treatment outcome and the second treatment outcome based on the multi-modal data and the determined cluster, treatment, flag and second flag of each test subject.

6. A system according to claim 1, the processing unit to execute the program code to cause the system to:
determine second multi-modal data associated with the subject, the second multi-modal data including second image data of the subject;
input the second multi-modal data to the first trained clustering model to determine a second cluster for the subject; and
input the second multi-modal data, the second cluster and the treatment to the second trained model, where the second trained model outputs a second probability associated with the treatment outcome in response to the input second multi-modal data, second cluster and treatment.

7. A system according to claim 1, the processing unit to execute the program code to cause the system to:
determine second multi-modal data associated with the subject, the second multi-modal data including second image data of the subject;
input the second multi-modal data to the first trained clustering model to determine a second cluster for the subject; and
input the second multi-modal data, the second cluster and a second treatment to the second trained model, where the second trained model outputs a second probability associated with the treatment outcome in response to the input second multi-modal data, second cluster and second treatment.

8. A method comprising:
determining multi-modal data associated with a subject, the multi-modal data including image data of the subject;
inputting the multi-modal data to a first trained clustering model to determine a cluster for the subject;
determining a proposed treatment for the subject; and
inputting the multi-modal data, the cluster and the treatment to a second trained model, where the second trained model outputs a probability associated with a treatment outcome in response to the input multi-modal data, cluster and treatment.

9. A method according to claim 8, wherein the probability is a probability that the treatment will result in the treatment outcome for the subject.

10. A method according to claim 9, wherein the second trained model outputs a second probability associated with a second treatment outcome in response to the input multi-modal data, cluster and treatment, and
wherein the second probability is a probability that the treatment will result in the second treatment outcome for the subject.

11. A method according to claim 8, wherein the first trained clustering model is trained by:
receiving training multi-modal data associated with each of a plurality of test subjects; an
training the first clustering model using the training multi-modal data, and
wherein the second trained model is trained by:
determining a cluster for each test subject based on the trained first model;
determining, for each test subject, a treatment and a flag associated with the treatment outcome; and
training the second model to output a probability for the treatment outcome based on the multi-modal data and the determined cluster, treatment and flag of each test subject.

12. A method according to claim 11, wherein the second trained model outputs a second probability associated with a second treatment outcome in response to the input multi-modal data, cluster and treatment,
wherein the probability is a probability that the treatment will result in the treatment outcome for the subject,
wherein the second probability is a probability that the treatment will result in the second treatment outcome for the subject, and
wherein the second trained model is trained by:
determining, for each test subject, a second flag associated with the second treatment outcome; and
training the second model to output probabilities for the treatment outcome and the second treatment outcome based on the multi-modal data and the determined cluster, treatment, flag and second flag of each test subject.

13. A method according to claim 8, further comprising:
determining second multi-modal data associated with the subject, the second multi-modal data including second image data of the subject;
inputting the second multi-modal data to the first trained clustering model to determine a second cluster for the subject; and
inputting the second multi-modal data, the second cluster and the treatment to the second trained model, where the second trained model outputs a second probability associated with the treatment outcome in response to the input second multi-modal data, second cluster and treatment.

14. A method according to claim 8, further comprising:
determining second multi-modal data associated with the subject, the second multi-modal data including second image data of the subject;
inputting the second multi-modal data to the first trained clustering model to determine a second cluster for the subject; and
inputting the second multi-modal data, the second cluster and a second treatment to the second trained model, where the second trained model outputs a second probability associated with the treatment outcome in response to the input second multi-modal data, second cluster and second treatment.

15. A non-transitory computer-readable medium storing program code executable to cause a computing system to:
determine multi-modal data associated with a subject, the multi-modal data including image data of the subject;
input the multi-modal data to a first trained clustering model to determine a cluster for the subject;
determine a proposed treatment for the subject; and
input the multi-modal data, the cluster and the treatment to a second trained model, where the second trained model outputs a probability associated with a treatment outcome in response to the input multi-modal data, cluster and treatment.

16. A medium according to claim 15, wherein the probability is a probability that the treatment will result in the treatment outcome for the subject.

17. A medium according to claim 16, wherein the second trained model outputs a second probability associated with a second treatment outcome in response to the input multi-modal data, cluster and treatment, and
wherein the second probability is a probability that the treatment will result in the second treatment outcome for the subject.

18. A medium according to claim 15, wherein the first trained clustering model is trained by:
receiving training multi-modal data associated with each of a plurality of test subjects; an
training the first clustering model using the training multi-modal data, and
wherein the second trained model is trained by:
determining a cluster for each test subject based on the trained first model;
determining, for each test subject, a treatment and a flag associated with the treatment outcome; and
training the second model to output a probability for the treatment outcome based on the multi-modal data and the determined cluster, treatment and flag of each test subject.

19. A medium according to claim 15, wherein the second trained model outputs a second probability associated with a second treatment outcome in response to the input multi-modal data, cluster and treatment,
wherein the probability is a probability that the treatment will result in the treatment outcome for the subject,
wherein the second probability is a probability that the treatment will result in the second treatment outcome for the subject, and
wherein the second trained model is trained by:
determining, for each test subject, a second flag associated with the second treatment outcome; and
training the second model to output probabilities for the treatment outcome and the second treatment outcome based on the multi-modal data and the determined cluster, treatment, flag and second flag of each test subject.

20. A medium according to claim 15, the program code executable to cause the computing system to:
determine second multi-modal data associated with the subject, the second multi-modal data including second image data of the subject;
input the second multi-modal data to the first trained clustering model to determine a second cluster for the subject; and
input the second multi-modal data, the second cluster and the treatment to the second trained model, where the second trained model outputs a second probability associated with the treatment outcome in response to the input second multi-modal data, second cluster and treatment.
